(19) United States
(12) Patent Application Publication
(10) Pub. No.: US 2026/0076713 A1
(43) Pub. Date: Mar. 19, 2026

(54) PERICARDIAL SPACE ACCESS

(71) Applicant: Edwards Lifesciences Corporation, Irvine, CA (US)

(72) Inventors: Leonardo Paim Nicolau Da Costa, Tampa, FL (US); Atiya Makhdoom Ahmad, Irvine, CA (US)

(21) Appl. No.: 19/401,809

(22) Filed: Nov. 26, 2025

Related U.S. Application Data
(63) Continuation of application No. PCT/US2024/031278, filed on May 28, 2024.
(60) Provisional application No. 63/505,674, filed on Jun. 1, 2023.

(51) Int. Cl. A61B 17/34; A61M 25/06; A61M 25/09; A61B 17/00

(52) U.S. Cl. CPC A61B 17/3421; A61B 17/3403; A61M 25/065; A61M 25/09041; A61B 2017/00044; A61M 2230/04

(57) ABSTRACT

A medical access system can comprise an inner needle assembly including an inner needle shaft configured to be slidably disposed at least partially within a delivery lumen of an outer needle assembly, the inner needle shaft comprising a narrower distal portion and a wider portion proximal of the narrower distal portion, and a transition segment between the narrower distal portion and the wider portion. A first delivery lumen within the inner needle shaft can extend from a proximal end of the inner needle shaft to a distal end opening at a distal end of the narrower distal portion, and a second delivery lumen within the inner needle shaft can extend from the proximal end to a distal opening associated with the transition segment.

### CROSS REFERENCE TO RELATED APPLICATIONS

This application is a continuation of International Patent Application No. PCT/US2024/031278, filed May 28, 2024, which claims the benefit of U.S. Patent Application No. 63/505,674, filed June 1, 2023, the disclosures of which are hereby expressly incorporated by reference herein in their entirety for all purposes.

### BACKGROUND

Access to the heart pericardial space through the pericardium can be difficult. The pericardial space can be a relatively narrow space. Force needed to puncture the pericardium, in combination with the narrowness of the pericardial space, can often result in overshooting the pericardial space. Overshooting the pericardial space can cause inadvertent entry into the adjacent and/or underlying heart chamber, such as a heart ventricle or atrium. Adding to the challenge of accessing the pericardial space, some patients may have little or no fluid within the pericardial space. Use of large standard sized needles to access the pericardial space may cause excessive bleeding upon withdrawal of the needle if the heart ventricle is punctured.

### SUMMARY

Described herein are methods and devices relating to medical access systems configured to provide access to the pericardial space. In some instances, a medical access system can comprise an outer needle assembly and an inner needle assembly having an inner needle shaft with a narrower distal portion and a wider portion proximal of the narrower distal portion. A transition segment can extend between the narrower distal portion and the wider portion. At least a portion of the inner needle shaft can be configured to be slidably disposed within a delivery lumen of the outer needle assembly. In some instances, the inner needle assembly can be advanced relative to the outer needle assembly to target positioning at least a portion of the narrower distal portion of the inner needle shaft into a heart pericardial space. In some instances, the inner needle assembly can be advanced relative to the outer needle assembly to position a distal opening on the transition segment of the inner needle shaft into the heart pericardial space. The distal opening on the transition segment can be positioned into the heart pericardial space while a portion of the narrower distal portion is positioned into a heart ventricle adjacent to the heart pericardial space. In some instances, a medical access system can comprise an outer needle assembly and an inner needle assembly with an inner needle shaft having a uniform or substantially uniform outer diameter. At least a portion of the inner needle shaft can be configured to be slidably disposed within a delivery lumen of the outer needle assembly. In some instances, the inner needle assembly can be advanced relative to the outer needle assembly to target positioning a distal end of the inner needle shaft into a heart pericardial space. In some instances, the outer needle assembly can be advanced relative to the inner needle assembly to position a distal end opening at a distal end of the outer needle shaft into the heart pericardial space. The distal end opening of the outer needle shaft can be positioned into the heart pericardial space while the distal end of the inner needle shaft is positioned into a heart ventricle adjacent to the heart pericardial space.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silico, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages, and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 shows an example of a medical access system deployed into a heart to provide access to a pericardial space of the heart in accordance with one or more examples.

FIG. 2 provides a side view of the medical access system of FIG. 1 in accordance with one or more examples.

FIGS. 3A and 3B provide side cross-sectional views of the deployment of the inner needle assembly of FIG. 2 into a pericardial space and deployment of a guidewire into the pericardial space using the inner needle assembly, respectively, in accordance with one or more examples.

FIGS. 4A and 4B provide side cross-sectional views of the deployment of a first guidewire into a heart ventricle using the inner needle assembly of FIG. 2 and deployment of a second guidewire into a pericardial space using the outer needle assembly of FIG. 2, respectively, in accordance with one or more examples.

FIG. 5 is a process flow diagram illustrating a procedure for accessing a heart pericardial space in accordance with one or more examples.

FIG. 6 provides a side view of a medical access system configured to provide access to a heart pericardial space in accordance with one or more examples.

FIGS. 7A and 7B provide side cross-sectional views of the deployment of the medical access system of FIG. 6 for accessing a heart pericardial space in accordance with one or more examples.

FIG. 8 is a process flow diagram illustrating a procedure for accessing a heart pericardial space in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Access to the cardiac pericardial space through the pericardium can be difficult. The pericardial space can be a relatively narrow space. Force needed to puncture the pericardium, including in combination with the narrowness of the pericardial space, can often result in overshooting the pericardial space and inadvertent entry into the adjacent heart chamber. For example, overshooting the pericardial space can result in inadvertent entry into the adjacent heart ventricle or heart atrium. Patients with little or no fluid within the pericardial space can make targeting the pericardial space more challenging, such as performing a dry tap. Use of standard gauge needles for accessing the pericardial space can result in excessive bleeding. For example, the large bore size of the standard gauge needles can result in creating a cut in the myocardium after withdrawal of the needle if the myocardium is pierced by the standard gauge needle, resulting in excessive bleeding through the cut. Excessive bleeding can lead to, for example, pericardial tamponade, shock and/or other severe complications.

Described herein are methods and devices relating to medical access systems configured to provide access to the pericardial space. In some instances, a medical access system can comprise an outer needle assembly and an inner needle assembly. The inner needle assembly can comprise an inner needle shaft extending distally from an inner needle hub. The inner needle shaft can comprise a narrower distal portion and a wider portion proximal of the narrower distal portion. A transition segment can extend between the narrower distal portion and the wider portion, for example joining the narrower distal portion and the wider portion. In some instances, the narrower distal portion can comprise a micropuncture needle. At least a portion of the inner needle shaft can be configured to be slidably disposed within the delivery lumen of the outer needle assembly. The inner needle assembly can be configured to be advanced relative to the outer needle assembly to target positioning at least a portion of the narrower distal portion into a heart pericardial space. In the case that the inner needle assembly overshoots the heart pericardial space, for example the narrower distal portion being positioned into a heart chamber, the inner needle assembly can be advanced further to position a distal opening on the transition segment into the heart pericardial space.

In some instances, a medical access system can comprise an outer needle assembly and an inner needle assembly. The inner needle assembly can comprise an inner needle shaft extending distally from an inner needle hub. In some instances, the inner needle shaft can have a uniform or substantially uniform lateral dimension, such as an outer diameter. In some instances, the inner needle shaft can comprise a micropuncture needle. At least a portion of the inner needle shaft can be configured to be slidably disposed within a delivery lumen extending through the outer needle assembly. The inner needle assembly can be advanced relative to the outer needle assembly to target positioning a distal end of the inner needle shaft into a pericardial space. In the case that a portion of the inner needle shaft is unintentionally advanced beyond the pericardial space, such as being deployed through the myocardium and into a heart chamber, the outer needle assembly can be advanced relative to the inner needle assembly to position a portion of the outer needle shaft into the pericardial space. For example, a distal end opening at a distal end of the outer needle shaft can be position into the pericardial space.

As used herein, a “delivery lumen” can refer to any number of lumens, channels, passages, and/or conduits through which a medical instrument and/or medical device can be advanced, including for example a working channel. For example, a “delivery lumen” as used herein can refer to a working channel through which a medical instrument and/or medical device can be advanced for positioning the medical instrument and/or medical device at a target site within a patient.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

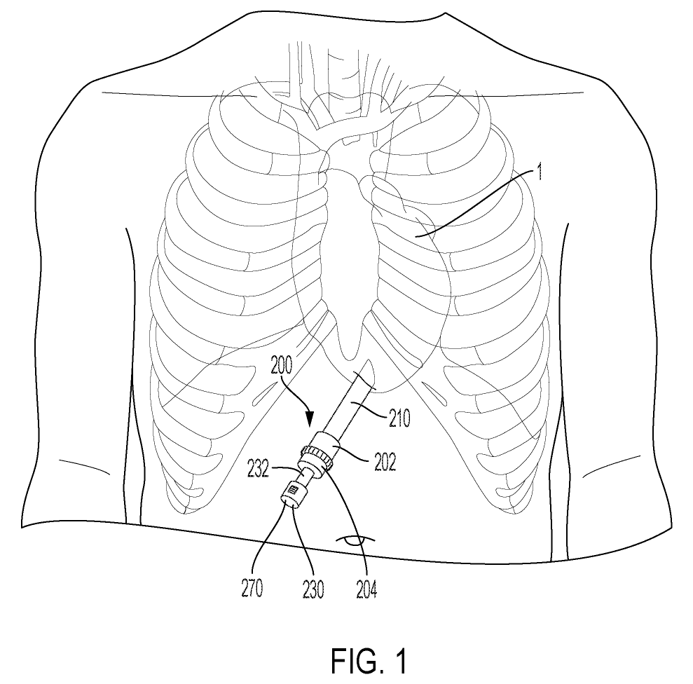

FIG. 1 shows an example of a medical access system 200 deployed into a heart 1 to provide access to a pericardial space of the heart 1. The medical access system 200 can provide percutaneous access into the pericardial space. The medical access system 200 can comprise an outer needle assembly 202 and an inner needle assembly 230. The inner needle assembly 230 can comprise an inner needle shaft 232 extending distally from an inner needle hub 270. In some instances, the inner needle assembly 230 can comprise a portion preloaded within the delivery lumen 218 of the outer needle assembly 202 while the outer needle assembly 202 is advanced into the chest cavity through an access point on the chest. In some instances, the outer needle assembly 202 can be inserted percutaneously through the access point on the chest. The inner needle shaft 232 can be configured to be slidably disposed within the outer needle assembly 202. At least a portion of the outer needle shaft 210 can be advanced into the chest cavity. After the outer needle shaft 210 is desirably positioned, inner needle assembly 230 can be deployed to provide access to the pericardial space.

In some instances, insertion of the medical access system 200 into the chest cavity can be made at various insertion sites on the chest. In some instances, access into the chest cavity can be made using a substernal, parasternal, or apical approach. For example, FIG. 1 shows using the medical access system 200 to enter the chest cavity using a substernal approach. In some instances, the medical access system 200 can be used to access the cardiothoracic cavity using a subxiphoid access approach for entry into the pericardial space. In some instances, a trans-apical approach can be used to access the cardiothoracic cavity for entering the pericardial space. Although methods and devices described herein refer primarily to access of the heart pericardial space, it will be understood that the methods and/or devices can be applied to controlled entry of any other vessels, chambers, lumen, and/or organ.

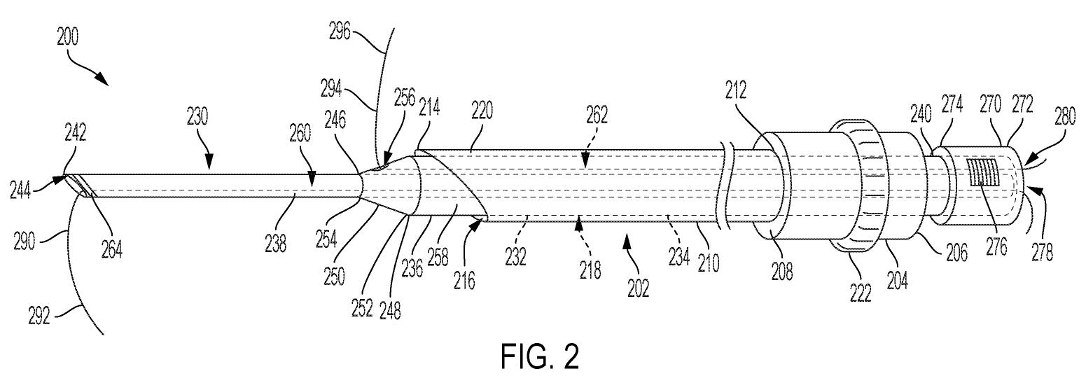

FIG. 2 is a side view of the medical access system 200 comprising the outer needle assembly 202 and the inner needle assembly 230. The outer needle assembly 202 can comprise an outer needle hub 204 and an outer needle shaft 210 extending distally from the outer needle hub 204. The delivery lumen 218 of the outer needle assembly 202 can extend through the outer needle hub 204 and the outer needle shaft 210. The inner needle assembly 230 can comprise an inner needle shaft 232 extending distally from an inner needle hub 270. At least a portion of the inner needle shaft 232 can be configured to be slidably received within the delivery lumen 218 of the outer needle assembly 202. The inner needle shaft 232 can comprise a narrower distal portion 238 and a wider portion 236 proximal of the narrower distal portion 238. A transition segment 250 can extend between the narrower distal portion 238 and the wider portion 236, for example joining the narrower distal portion 238 and the wider portion 236. A lateral dimension, such as an outer diameter, of the narrower distal portion 238 can be shorter than that of the wider portion 236. The lateral dimension can be perpendicular or substantially perpendicular to a longitudinal axis of the inner needle shaft 232.

In some instances, the wider portion 236 can comprise a proximal portion 234 of the inner needle shaft 232. In some instances, the wider portion 236 can comprise the portion of the inner needle shaft 232 proximal of the transition segment 250 and distal of the distal end 274 of the inner needle hub 270. For example, the wider portion 236 can extend from the transition segment 250 to a proximal end 240 of the inner needle shaft 232. In some instances, the wider portion 236 can extend from the proximal end 240 that of the inner needle shaft 232 to a proximal end 252 of the transition segment 250. For example, a distal end 248 of the wider portion 236 can be adjacent to and in contact with the proximal end 252 of the transition segment 250. In some instances, an outer diameter of the wider portion 236 can be uniform or substantially uniform along a longitudinal dimension thereof, including from the proximal end 240 of the inner needle shaft 232 to the distal end 248.

In some instances, the narrower distal portion 238 can comprise the portion of the inner needle shaft 232 distal of the transition segment 250. In some instances, the narrower distal portion 238 can extend from the transition segment 250 to a distal end 242 of the inner needle shaft 232, such as from a distal end 254 of the transition segment 250 to the distal end 242 of the inner needle shaft 232. For example, a proximal end 246 of the narrower distal portion 238 can be adjacent to and in contact with the distal end 254 of the transition segment 250. The distal end 242 of the inner needle shaft 232 can be a distal end of the narrower distal portion 238. In some instances, the distal end 242 can comprise at least a portion that is sharp and configured to pierce tissue to allow advancement of the inner needle shaft 232 through tissue. For example, the distal end 242 can be configured to pierce the pericardium to allow advancement of the inner needle shaft 232 into the pericardial space. In some instances, a sidewall 258 of the inner needle shaft 232 can define a shaft with the narrower distal portion 238, the wider portion 236, and the transition segment 250 extending distally from the wider portion 236 to the narrower distal portion 238. In some instances, an outer diameter of the distal narrower portion 238 can be uniform or substantially uniform along a longitudinal dimension thereof, including from the proximal end 246 to the distal end 242.

In some instances, the narrower distal portion 238 can comprise a lateral dimension, such as an outer diameter, smaller than that of a standard gauge needle. In some instances, the lateral dimension of the narrower distal portion 238 can be the same or similar to that of a micropuncture needle. In some instances, the narrower distal portion 238 can comprise a micropuncture needle. In some instances, the lateral dimension of the narrower distal portion 238 can be the same as or similar to that of a 22-gauge (G) needle. The narrower distal portion 238 can comprise a 22-gauge (G) micropuncture needle. In some instances, the lateral dimension of the narrower distal portion 238 can be smaller than that of a standard gauge needle, including between that of a standard gauge needle and that of a micropuncutre needle, such as that of an 18-gauge (G) needle and the 22-gauge (G) micropuncture needle. In some instances, the lateral dimension of the narrower distal portion 238 can be between about 0.6 millimeters (mm) to about 1.2 millimeters (mm), including about 0.7 millimeters (mm) to about 1.1 millimeters (mm), and about 0.7 millimeters (mm) to about 1.0 millimeters (mm).

In some instances, the transition segment 250 can comprise a tapered transition segment. In some instances, the transition segment 250 can have a tapered profile. The inner needle shaft 232 can have a tapered transition comprising a profile that tapers from the wider portion 236 to the narrower distal portion 238. The transition segment 250 can taper along a direction parallel or substantially parallel to the longitudinal axis of the inner needle shaft 232, such as extending from the proximal end 252 to an opposing portion on the distal end 254. For example, the transition segment 250 can assume a tapered profile extending from the wider portion 236 to the narrower distal portion 238. The tapered profile can comprise a linear taper and/or curved taper, including a convex curvature and/or concave curvature. In some instances, the transition segment 250 can taper linearly or substantially linearly from the proximal end 252 to the distal end 254. In some instances, the transition segment 250 can taper along a concave curvature or convex curvature from the proximal end 252 to the distal end 254.

In some instances, the transition segment 250 can extend circumferentially around the inner needle shaft 232. In some instances, the tapered transition segment can extend circumferentially around the inner needle shaft 232. The tapered transition may or may not be uniform around the circumference of the inner needle shaft 232. In some instances, the transition segment 250 can form a truncated cone shape, such as shown in FIG. 2. In some instances, the transition segment 250 can have a non-uniform tapered profile around the circumference of the inner needle shaft 232, such as having a larger and/or steeper taper on a portion having a distal opening 256 than an opposing portion. Alternatively, the transition segment 250 can extend partially around the circumference of the inner needle shaft 232. For example, the tapered transition segment can extend partially around the circumference of the inner needle shaft 232. In some instances, the transition segment 250, including the tapered transition segment, can extend around a first circumferential portion of the inner needle shaft 232. A second circumferential portion of the inner needle shaft 232 can comprise a linear or substantially linear profile. For example, the inner needle shaft 232 can have a tapered transition segment around a first partial circumferential portion, and a linear or substantially linear profile around a second partial circumferential portion, including a remainder of the circumference of the inner needle shaft. In some instances, where the wider portion 236 and the narrower distal portion 238 meet, join and/or merge together around the remainder of the circumference of the inner needle shaft 232 can have the linear or substantially linear profile.

The inner needle assembly 230 can comprise a first delivery lumen 260 and a second delivery lumen 262. The first delivery lumen 260 can comprise a portion extending through the narrower distal portion 238. For example, the first delivery lumen 260 can comprise a portion extending through the inner needle shaft 232, including from the proximal end 240 of the inner needle shaft 232 to the distal end 242 of the inner needle shaft 232. The first delivery lumen 260 can be in fluid communication with a distal end opening 244 at the distal end 242 of the inner needle shaft 232, such as at the distal end of the narrower distal portion 238. In some instances, the first delivery lumen 260 can extend through the inner needle hub 270 and the inner needle shaft 232 to the distal end 242 of the inner needle shaft 232. The second delivery lumen 262 can comprise a portion extending through the wider portion 236 of the inner needle shaft 232. In some instances, the second deliver lumen 262 can comprise a portion extending from the proximal end 240 of the inner needle shaft 232 to the distal opening 256 associated with the transition segment 250. In some instances, the second delivery lumen 262 can extend through the inner needle hub 270 and the portion of the inner needle shaft 232 proximal of the narrower distal portion 238, such as through the wider portion 236 of the inner needle shaft 232 to the distal opening 256. In some instances, the distal opening 256 can be on the transition segment 250, such as extending through a sidewall portion 258 of the inner needle shaft 232 defining the transition segment 250. As described herein, the transition segment 250 can comprise a tapered transition segment. In some instances, the distal opening 256 can be on the tapered transition segment. For example, the distal opening 256 can be on a tapered portion of the transition segment 250. FIG. 2 shows the transition segment 250 comprising a tapered profile extending circumferentially around the inner needle shaft 232. In alternative instances, a first circumferential portion can comprise the transition segment 250 having a tapered profile and a second circumferential portion having a linear or substantially linear profile. The distal opening 256 can be on the first circumferential portion, including on the tapered portion. The first and second delivery lumens 260, 262 can comprise at least a portion extending alongside one another and/or be parallel or substantially parallel to the longitudinal axis of the inner needle shaft 232. In some instances, the inner needle shaft 232 can be a dual lumen shaft.

The first and/or second delivery lumens 260, 262 can be configured to allow advancement therethrough of a respective guidewire. In some instances, at least a portion of a guidewire having a lateral dimension, such as a diameter, of about 0.25 millimeters (mm) to about 0.50 millimeters (mm), including about 0.35 millimeters (mm) to about 0.45 millimeters (mm) can be advanced through the first and/or second delivery lumens 260, 262. In some instances, at least a portion of a guidewire having a diameter of about 0.010 inches (in) to about 0.020 inches (in), including about 0.014 inches (in) to about 0.018 inches (in) can be advanced through the first and/or second delivery lumens 260, 262. In alternative instances, the guidewire can have a larger or smaller diameter than the diameters described herein. For example, a first guidewire 290 can be deployed through the first delivery lumen 260. A second guidewire 294 can be deployed through the second delivery lumen 262. Deployment of the guidewires can facilitate secure access to the pericardial space. After secure access to the pericardial space is achieved, medical therapy can be delivered through the first delivery lumen 260 or second delivery lumen 262, including injection into and/or withdrawal from the pericardial space of any number of different fluids. In some instances, imaging contrast dye agents can be delivered. In some instances, one or more therapeutic agents can be delivered.

Referring again to FIG. 2, the outer needle assembly 202 can comprise a delivery lumen 218 extending through the outer needle assembly 202, including through the outer needle hub 204 and the outer needle shaft 210. The outer needle assembly 202 can comprise an outer needle shaft 210 extending distally from an outer needle hub 204. In some instances, the outer needle shaft 210 can comprise a lateral dimension, such as an outer diameter, the same as or similar to that of a standard gauge needle. The lateral dimension can be perpendicular or substantially perpendicular to a longitudinal axis of the outer needle shaft 210. In some instances, the outer needle shaft 210 can comprise a lateral dimension that is the same as or similar to that of a standard gauge needle. In some instances, an outer diameter of the outer needle shaft 210 can be uniform or substantially uniform along a longitudinal dimension of the outer shaft 210, such as from a proximal end 212 to a distal end 214 of the outer needle shaft 210. In some instances, the distal end 214 can comprise at least a portion that is sharp and configured to pierce tissue to allow advancement of the outer needle shaft 210 through tissue, as to allow the outer needle shaft 210 to navigate through a percutaneous access route. For example, a sidewall 220 of the outer needle shaft 210 can define a shaft having the same or similar outer diameter along an entire or substantially entire length of the outer needle shaft 210 distal of the distal end 208 of the outer needle hub 204. In some instances, the outer needle shaft 210 can comprise a standard gauge needle. In some instances, the outer needle shaft can comprise a lateral dimension, such as an outer diameter, that is the same as or similar to that of an 18-gauge (G) needle. The outer needle shaft 210 can comprise an 18-gauge (G) needle. In some instances, the lateral dimension of the outer needle shaft 210 can be larger than that of the standard gauge needle, such as the 18-gauge (G) needle. In some instances, the outer needle shaft 210 can comprise a lateral dimension, such as an outer diameter that is about 1.0 millimeters (mm) to about 1.6 millimeters (mm), including about 1.0 millimeters (mm) to about 1.5 millimeters (mm), and about 1.2 millimeters (mm) to about 1.4 millimeters (mm).

At least a portion of the inner needle shaft 232 can be slidably disposed in the delivery lumen 218 of the outer needle assembly 202. As described in further detail herein, the inner needle shaft 232 can be preloaded within the outer needle assembly 202 while navigating the medical access system 200 to a target position. The inner needle assembly 230 can be configured to be subsequently advanced relative to the outer needle assembly 202 to target positioning at least a portion of the narrower distal portion 238 distally of the distal end 214 of the outer needle assembly and into a heart pericardial space. In some instances, the inner needle shaft 232 can be advanced until the distal end 274 of the inner needle hub 270 contacts the proximal end 206 of the outer needle hub 204. In the case that the inner needle assembly 230 overshoots the heart pericardial space, for example the narrower distal portion 238 being positioned into a heart chamber, inner needle assembly 230 can be advanced further to position the distal opening 256 on the transition segment 250 into the heart pericardial space. After the narrower distal portion 238 is positioned into an adjacent heart ventricle or atrium, the inner needle assembly 230 can be advanced further to position the distal opening 256 into the heart pericardial space.

In some instances, the medical access system 200 can be configured to provide controlled advancement and/or retraction of the inner needle assembly 230 relative to the outer needle assembly 202. For example, the outer needle assembly 202, such as the outer needle hub 204, can comprise an advancement and retraction control 222 configured to be in contact with the inner needle shaft 232 and provide controlled advancement and/or retraction of the inner needle shaft 232. In some instances, the advancement and retraction control 222 can provide incremental advancement and/or retraction of the inner needle shaft 232, such as a ratchet mechanism. For example, an operator, such as a surgeon can turn a knob, dial, and/or lever of the advancement and retraction control 222 to incrementally advance and/or retract the inner needle shaft 232 relative to the outer needle assembly 202, including the outer needle shaft 210. In some instances, the advancement and retraction control 222 can be configured to provide visual (e.g., numbered scale), audible and/or tactile feedback, including after each incremental advancement and/or retraction, such as after every 1 millimeter (mm) of movement. In some instances, at least a portion of the outer needle shaft 210 and/or inner needle shaft 232 can be radiopaque. For example, the outer needle shaft 210 and the inner needle shaft 232 can be radiopaque to allow visualization of their respective positions, facilitating placement of the respective shafts. In some instances, radiopaque needle shafts having smaller diameters can be more difficult to visualize. The advancement and retraction control 222 can facilitate controlled advancement and/or retraction of the inner needle shaft 232 comprising a micropuncture needle.

In some instances, the inner needle hub 270 can comprise a first port 278, such as on a proximal end 272 of the inner needle hub 270, configured to mate with a first syringe (not shown). In some instances, the first port 278 can be in fluid communication with the first delivery lumen 260. For example, the first syringe can draw pericardial fluid from a pericardial space through the first delivery lumen 260 and the first port 278 while at least a portion of the narrower distal portion 238 is positioned in the pericardial space. Alternatively, the first syringe can draw blood from a heart ventricle or heart atrium through the first delivery lumen 260 and the first port 278 if at least a portion of the narrower distal portion 238 is positioned in the heart ventricle or heart atrium. In some instances, the inner needle hub 270 can comprise a second port 280 configured to mate with a second syringe (not shown). The second port 280 can be in fluid communication with the second delivery lumen 262. For example, the second syringe can draw pericardial fluid from a pericardial space through the distal opening 256 and the second delivery lumen 262 while the second distal opening 256 is positioned in the pericardial space. In some instances, the first syringe can be mated with the first port 278 while the distal end opening 244 is in the heart ventricle or heart atrium and the distal opening 256 is in the pericardial space so that blood can be drawn by the first syringe. In some instances, allowing withdrawal of pericardial fluid and/or blood by the syringes can facilitate confirmation of a position of the inner needle shaft 232.

In some instances, the inner needle shaft 232 can comprise an electrode 264 configured to provide an electrical signal indication when the electrode 264 and/or shaft 232 are in contact with a heart myocardium. The electrode 264 can be configured to provide electrical feedback indicating that the inner needle shaft 232 has been inserted further than desired. For example, the electrode 264 can comprise at least a portion associated with, for example on and/or coupled to, the distal end 242 of the inner needle shaft 232. The electrode 264 can be configured to provide an electrical signal indication when a portion of the inner needle shaft 232, including the distal end 242, and/or the electrode 264, is in contact with the heart myocardium. The electrode 264 can be in electrical communication with an output on the inner needle assembly 230, such as the output contact 276 of the inner needle hub 270, configured to provide the electrical measurements to an electrocardiogram machine. The electrocardiogram can receive the measurements through the output contact 276, such as via one or more leads. For example, an ST elevation displayed by a user interface of the electrocardiogram machine can indicate to the operator that the inner needle shaft 232 has been inserted too far.

In some instances, the outer needle assembly 202 can comprise an electrode (not shown) configured to provide an electrical signal indication when contact is made with a heart myocardium. For example, the outer needle shaft 210, including the distal end 214 of the outer needle shaft 210, can comprise an electrode having one or more characteristics of the electrode 264 of the inner needle assembly 230. The outer needle hub 204 can comprise an output contact (not shown) having one or more characteristics of the output contact 276 of the inner needle assembly 230.

In some instances, the inner needle assembly 230 can comprise one or more pressure sensors (not shown). For example, one or more pressure sensors can be associated with respective portions of the proximal portion 234, including the proximal end 240, and/or distal end 242 of the inner needle shaft 232. The pressure sensors can be coupled to, including mechanically and/or electrically, to a pressure sensor output on the inner needle hub 270. The pressure sensors can allow reading of pressures the inner needle shaft 232 is exposed to, for example facilitating determining a location of the inner needle shaft 232 to thereby facilitate navigation of the inner needle shaft 232 to the target location.

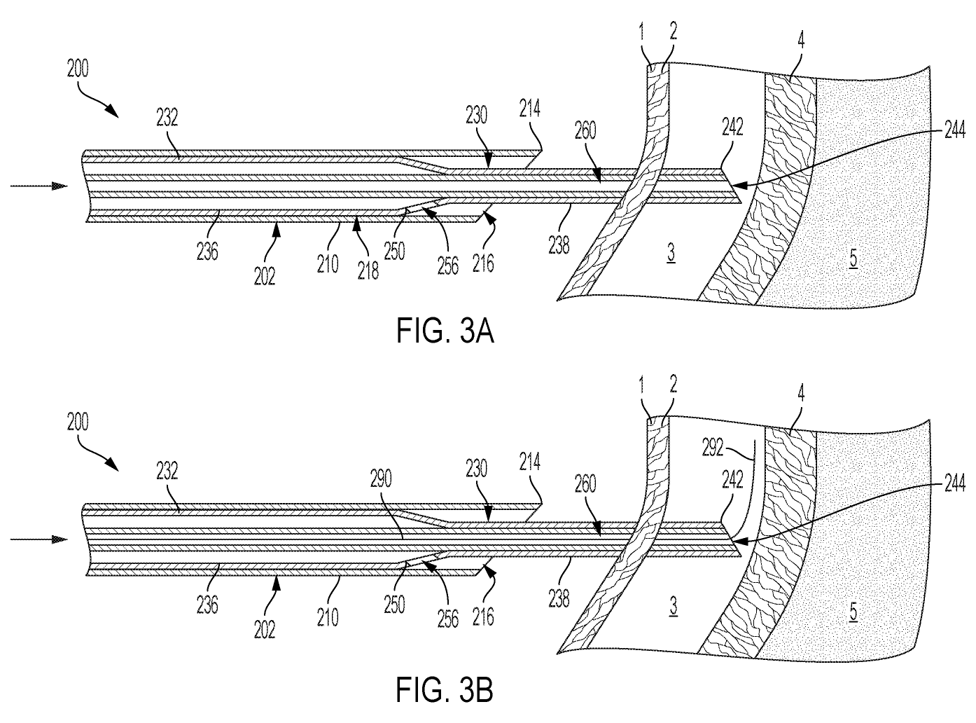

FIGS. 3A and 3B are side cross-sectional views showing deployment of a first guidewire 290 into a pericardial space 3 of a heart 1 using the medical access system 200 described with reference to FIG. 2. As described herein, at least a portion of the inner needle shaft 232 can be slidably disposed in the delivery lumen 218 of the outer needle assembly 202 while the outer needle shaft 210 is navigated to a target position. For example, the inner needle shaft 232 can be preloaded within the delivery lumen 218 of the outer needle assembly 202 with the distal end 242 of the inner needle shaft 232 within the delivery lumen 218 and proximal of the distal end 214 of the outer needle shaft 210. In some instances, navigation of the outer needle shaft 210 to the target position can be echo-guided and/or assisted using fluoroscopy. The distal end 214 of the outer needle shaft 210 can be positioned proximate and/or adjacent to an outer surface of a pericardium 2. In some instances, the distal end 214 can be at a position that is spaced from and not in contact with the outer surface of the pericardium 2. For example, the distal end 214 can be maintained at its position spaced from the outer surface of the pericardium 2 while the inner needle shaft 232 is deployed through the distal end opening 216 of the outer needle shaft 210. Alternatively, the distal end 214 of the outer needle shaft 210 can be over and in contact with the outer surface of the pericardium 2 while the inner needle shaft 232 is deployed through the distal end opening 216 of the outer needle shaft 210.

After the outer needle shaft 210 is desirably positioned, the inner needle assembly 230 can be advanced relative to the outer needle assembly 202 to position a portion of the inner needle shaft 232 through the distal end opening 216 of the outer needle shaft 210. Referring to FIG. 3A, the narrower distal portion 238 can be inserted through the pericardium 2 and positioned into the pericardial space 3 such that the distal end opening 244 of the inner needle shaft 232 can be disposed within the pericardial space 3. Advancement of the inner needle shaft 232 through delivery lumen 218 of the outer needle shaft 210 can experience no or reduced friction, facilitating controlled advancement of the inner needle shaft 232 to position the narrower distal portion 238 into the pericardial space 3. The outer needle shaft 210 can provide support and/or guidance for the inner needle shaft 232 during advancement of the inner needle shaft 232. Advancement of the inner needle shaft 232 slidably disposed within the outer needle assembly 202 can provide improved tactile feedback for controlled insertion of the narrower distal portion 238 into the pericardial space 3.

Referring to FIG. 3B, after the narrower distal portion 238 is desirably positioned, the first guidewire 290 can be deployed through the first delivery lumen 260 after the narrower distal portion 238 is positioned into the pericardial space 3. A portion of the first guidewire 290 can be advanced through the first delivery lumen 260 of the inner needle assembly 230 and into the pericardial space 3 through the distal end opening 244 disposed within the pericardial space 3. For example, a distal portion 292 of the first guidewire 290 can be positioned into the pericardial space 3. The first guidewire 290 can be deployed to facilitate securely positioning the narrower distal portion 238 within the pericardial space 3.

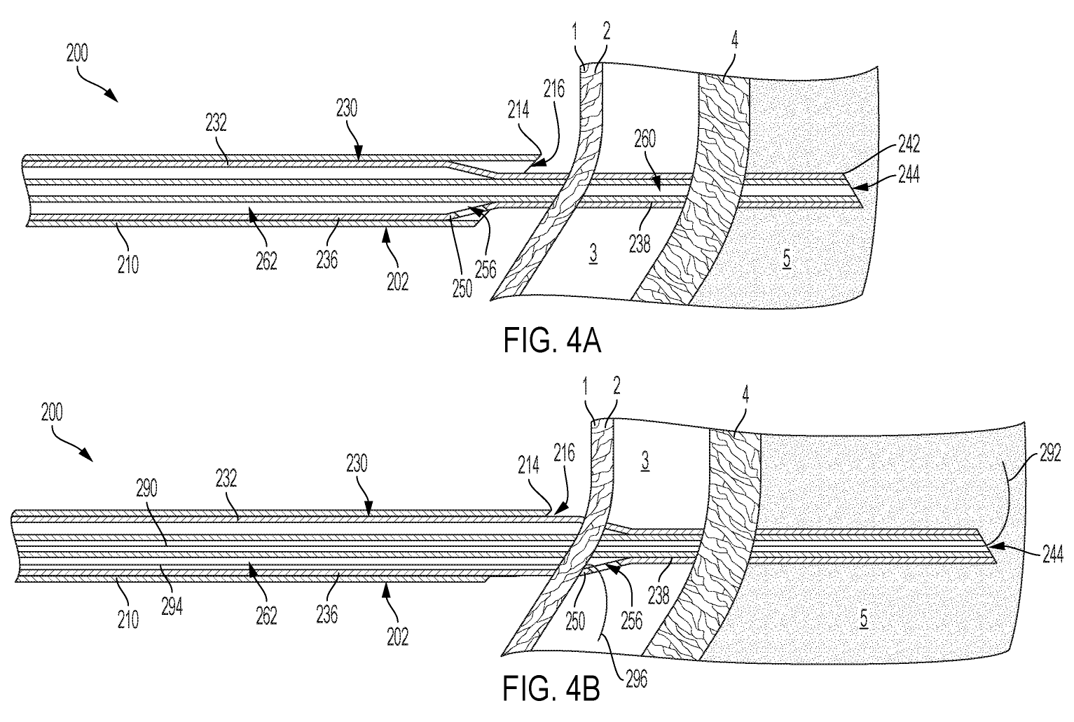

FIGS. 4A and 4B are side cross-sectional views showing deployment of a first guidewire 290 into a heart chamber 5 and a second guidewire 294 into a pericardial space 3 of a heart 1 using the medical access system 200 described with reference to FIG. 2. The heart chamber 5 can be a heart ventricle or a heart atrium. Referring to FIG. 4A, the narrower distal portion 238 is shown as being positioned into the heart chamber 5. As described herein, positioning the narrower distal portion 238 into the pericardial space 3 may be difficult. The narrower distal portion 238 can be advanced further than desired, overshooting the pericardial space 3. A portion of the narrower distal portion 238 can be positioned unintentionally through the myocardium 4 such that the distal end opening 244 at the distal end 242 of the inner needle shaft 232 is disposed in the heart chamber 5. For example, at least a portion of the inner needle shaft 232 can be preloaded within the outer needle assembly 202, such as while the outer needle shaft 210 is advanced along an anatomical pathway. A portion of the inner needle shaft 232 can be carried within the delivery lumen 218 of the outer needle assembly 202 such that the distal end 242 of the inner needle shaft 232 can be proximal of the distal end 214 of the outer needle shaft 210. The distal end 214 of the outer needle shaft 210 can be proximate to or on an externally oriented surface of the pericardium 2. The inner needle assembly 230 can then be advanced relative to the outer needle assembly 202 to position a portion of the inner needle shaft 232 through the distal end opening 216 of the outer needle shaft 210.

In the case where the narrower distal portion 238 of the inner needle shaft 232 overshoots the intended target position in the pericardial space 3, the inner needle shaft 232 can be advanced further relative to the outer needle shaft 210 to position at least a portion of the transition segment 250 into the pericardial space 3. Referring to FIG. 4B, the inner needle assembly 230 can be advanced further to position the distal opening 256 on the transition segment 250 into the pericardial space 3. The inner needle shaft 232 does not need to be retracted. For example, a position of the outer needle shaft 210 can be maintained while the inner needle shaft 232 is advanced. Bleeding due to puncturing of the myocardium by the narrower distal portion 238 can be reduced compared to that caused by larger bore needles and/or the wider portion 236.

In some instances, the first guidewire 290 can be deployed through the first delivery lumen 260 into the heart chamber 5. A portion of the first guidewire 290 can be advanced through the first delivery lumen 260 of the inner needle assembly 230 and into the heart chamber 5 through the distal end opening 244 disposed within the heart chamber 5. For example, a distal portion 292 of the first guidewire 290 can be disposed in the heart chamber 5. The second guidewire 294 can be deployed through the second delivery lumen 262 of the inner needle assembly 230 such that a portion of the second guidewire 294 can be positioned into the pericardial space 3. A portion of the second guidewire 294 can be advanced through the second delivery lumen 262 and into the pericardial space 3 through the distal opening 256 on the transition segment 250 disposed within the pericardial space 3. A distal portion 296 of the second guidewire 294 can be disposed in the pericardial space 3. Positioning the second guidewire 294 into the pericardial space 3 can facilitate securing the position of the distal opening 256 in the pericardial space 3. The first guidewire 290 can be deployed to facilitate securely positioning the narrower distal portion 238 within the heart chamber 5, thereby facilitate secure positioning of the distal opening 256 into the pericardial space 3. Alternatively, the first guidewire 290 may not be deployed. For example, the second guidewire 294 can be deployed through the second delivery lumen 262 into the pericardial space 3 without deploying the first guidewire 290 through the first delivery lumen 260 into the heart chamber 5.

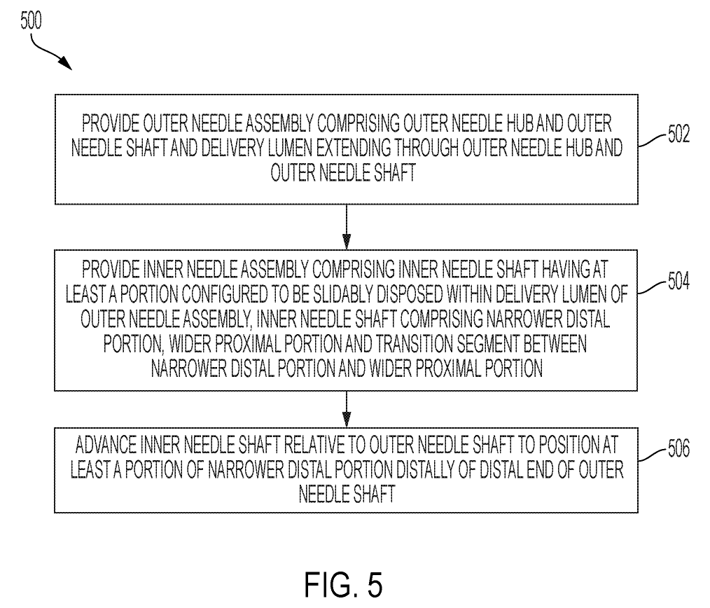

FIG. 5 is a process flow diagram illustrating an example of a procedure 500 for accessing a heart pericardial space. In block 502, the process can involve providing an outer needle assembly comprising an outer needle hub and an outer needle shaft extending distally from the outer needle hub. A delivery lumen can extend through the outer needle assembly, including the outer needle hub and the outer needle shaft. In block 504, the process can involve providing an inner needle assembly comprising an inner needle shaft with a narrower distal portion and a wider portion proximal of the narrower distal portion, and a transition between the narrower distal portion and the wider portion. At least a portion of the inner needle shaft can be slidably received within the delivery lumen of the outer needle assembly. In block 506, the process can involve advancing the inner needle shaft relative to the outer needle shaft to position at least a portion of the narrower distal portion distally of a distal end of the outer needle shaft.

In some instances, advancing the inner needle shaft relative to the outer needle shaft can comprise positioning at least a portion of the narrower distal portion into the heart pericardial space. For example, positioning at least a portion of the narrower distal portion distally of the distal end of the outer needle shaft can comprise positioning at least a portion of the narrower distal portion into the heart pericardial space.

In some instances, the process can involve advancing the outer needle assembly to a target position. For example, advancing the outer needle assembly to a target position can comprise advancing the outer needle shaft along an anatomical pathway to position the distal end of the outer needle shaft at a desired position. In some instances, advancing the outer needle assembly to the target position can comprise positioning the distal end of the outer needle shaft proximate and/or adjacent to an outer surface of a pericardium. Advancing the outer needle assembly to the target position can comprise positioning the distal end of the outer needle shaft at a position spaced from and not in contact with the outer surface of the pericardium. Alternatively, advancing the outer needle assembly to the target position can comprise positioning the distal end of the outer needle shaft over and in contact with the outer surface of the pericardium.

In some instances, at least a portion of the inner needle shaft can be slidably received within the delivery lumen of the outer needle assembly, for example preloaded within the delivery lumen, while the outer needle assembly is advanced along the anatomical pathway. For example, the outer needle shaft can be advanced along an anatomical pathway to position the distal end of the outer needle shaft at the target position, while at least a portion of the inner needle shaft is preloaded through the delivery lumen of the outer needle assembly. A distal end of the inner needle shaft can be within the delivery lumen, such as proximal of the distal end of the outer needle shaft, while the inner needle shaft is preloaded within the outer needle assembly. Alternatively, the inner needle shaft can be advanced through the delivery lumen of the outer needle assembly after the outer needle assembly is positioned at the target position.

In some instances, advancing the inner needle shaft relative to the outer needle shaft can comprise maintaining a position of the outer needle shaft while the inner needle shaft is deployed through a distal end opening at the distal end of the outer needle shaft. For example, the distal end of the outer needle shaft can be maintained at its position spaced from, or over and in contact with, the outer surface of the pericardium, while the inner needle shaft is deployed through the distal end opening of the outer needle shaft.

In some instances, providing the inner needle assembly can comprise providing a first delivery lumen extending through the inner needle shaft and in fluid communication with a distal end opening on a distal end of the inner needle shaft. The distal end of the inner needle shaft can be a distal end of the narrower distal portion. Providing the inner needle assembly can comprise providing a second delivery lumen extending through the wider portion of the inner needle shaft and in fluid communication with a distal opening on the transition segment of the inner needle shaft. In some instances, advancing the inner needle shaft relative to the outer needle shaft can comprise positioning the distal end opening on the distal end of the inner needle shaft into the heart pericardial space. In some instances, the process can involve deploying a portion of a first guidewire into the heart pericardial space through the first delivery lumen while at least a portion of the narrower distal portion and the distal end opening are disposed in the heart pericardial space. For example, at least a portion of the first guidewire can be advanced through the first delivery lumen and the distal end opening to position a portion of the first guidewire into the heart pericardial space. Positioning the portion of the first guidewire into the heart pericardial space can facilitate secure positioning of the narrower distal portion in the heart pericardial space.

Alternatively, advancing the inner needle shaft relative to the outer needle shaft can comprise positioning at least a portion of the narrower distal portion into a heart chamber, including a heart ventricle or heart atrium, for example positioning the distal end opening on the distal end of the inner needle shaft into the heart ventricle or atrium. In some instances, advancing the inner needle shaft can comprise overshooting the pericardial space such that a portion of the inner needle shaft is unintentionally positioned into the heart ventricle or atrium adjacent to the pericardial space. For example, the narrower distal portion can be advanced into the heart ventricle or atrium. In some instances, advancing the inner needle shaft relative to the outer needle shaft can comprise positioning the distal opening on the transition segment in the heart pericardial space. The process can involve advancing the inner shaft until the distal opening on the transition segment is disposed in the heart pericardial space, if the distal end opening at the distal end of the narrower distal portion overshoots the heart pericardial space. The position of the outer needle shaft can be maintained while the narrower distal portion is advanced, including while positioning the distal opening on the transition segment into the heart pericardial space.

In some instances, the process can involve deploying a second guidewire into the heart pericardial space while the distal opening on the transition segment is disposed in the heart pericardial space. For example, the process can involve advancing a portion of a second guidewire through the second delivery lumen and the distal opening on the transition segment into the pericardial space, while the distal opening on the transition is disposed in the heart pericardial space. In some instances, the process can involve deploying the first guidewire into the heart ventricle or atrium through the first delivery lumen while at least a portion of the narrower distal portion is disposed within the heart ventricle or atrium. For example, a portion of the first guidewire can be advanced through the first delivery lumen into the heart ventricle or atrium, while the distal end opening on the distal end of the narrower distal portion is positioned in the heart ventricle or atrium and the distal opening on the transition segment is positioned in the heart pericardial space. In some instances, the first guidewire may not be deployed into the heart ventricle or atrium.

In some instances, the process can involve mating a first syringe to a first port on the inner needle hub, the first port being in fluid communication with the first delivery lumen of the inner needle assembly to allow the first syringe to draw pericardial fluid from the heart pericardial space while the narrower distal portion and the distal end opening of the inner needle shaft is positioned in the pericardial space, or to draw blood from the heart ventricle or atrium while at least a portion of the narrower distal portion and the distal end opening of the inner needle shaft is positioned in the heart ventricle or atrium. In some instances, the process can involve mating a second syringe to a second port on the inner needle hub, the second port being in fluid communication with the second delivery lumen of the inner needle assembly to allow the second syringe to draw pericardial fluid from a pericardial space while the distal opening on the transition segment is positioned in the pericardial space. In some instances, one or more delivery lumens of the inner needle assembly can be used to deliver one or more agents to a target location, including a target location in the pericardial space. For example, contrast solution can be delivered through the one or more delivery lumens of the inner needle assembly to the pericardial space, such as for facilitating imaging procedures. In some instances, the process can involve contacting an electrode at the distal end of the narrower distal portion to a heart myocardium. Contacting the electrode to the heart myocardium can result in generating an electrical signal indication that the inner needle assembly has been inserted too far.

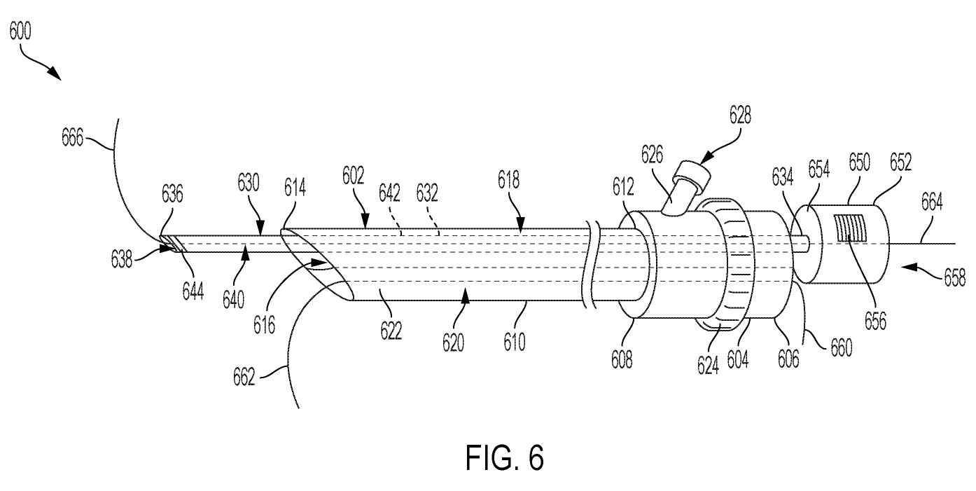

FIG. 6 is a side view of an example of a medical access system 600 configured to provide access to a pericardial space. The medical access system 600 can comprise an outer needle assembly 602 and an inner needle assembly 630. The outer needle assembly 602 can comprise an outer needle hub 604 and an outer needle shaft 610 extending distally from the outer needle hub 604. The inner needle assembly 630 can comprise an inner needle shaft 632 extending distally from an inner needle hub 650. In some instances, the outer needle assembly 602 can comprise a first delivery lumen 618 and a second delivery lumen 620 extending through the outer needle assembly 602. In some instances, the first and second delivery lumens 618, 620 can extend alongside one another through the outer needle assembly 602. At least a portion of the inner needle shaft 632 can be configured to be slidably received within the first delivery lumen 618 extending through the outer needle assembly 602. As described in further detail herein, in some instances, a guidewire can be advanced through the second delivery lumen 620. Alternatively, the outer needle assembly 602 can have a single delivery lumen extending through the needle assembly such that the guidewire can be advanced through the same delivery lumen through which the inner needle shaft 632 is slidably disposed.

In some instances, the inner needle shaft 632 can have a uniform or substantially uniform lateral dimension, such as a diameter. The lateral dimension can be perpendicular or substantially perpendicular to a longitudinal axis of the inner needle shaft 632. For example, the inner needle shaft 632 can have the same or similar outer diameter along an entire or substantially entire longitudinal dimension, such as length, of the inner needle shaft 632. The inner needle shaft 632 can have the same outer diameter along the entire length extending distally of a distal end 654 of the inner needle hub 650, including from a proximal end 634 to a distal end 636 of the inner needle shaft 632. The longitudinal dimension can be a dimension parallel or substantially parallel to the longitudinal axis of the inner needle shaft 632. In some instances, a sidewall 642 of the inner needle shaft 632 can define a shaft having the same or similar outer diameter along an entire or substantially entire length of the inner needle shaft 632 distal of the distal end 654 of the inner needle hub 650.

In some instances, the inner needle shaft 632 can comprise a needle. In some instances, the lateral dimension, such as the outer diameter, of the inner needle shaft 632 can be smaller than that of a standard gauge needle. In some instances, the inner needle shaft 632 can have a lateral dimension, such as an outer diameter, that is the same or similar to that of a micropuncture needle. In some instances, the inner needle shaft 632 can comprise a micropuncture needle. In some instances, the outer diameter of the inner needle shaft 632 can be the same as or similar to that of a 22-gauge (G) needle. The inner needle shaft 632 can comprise a 22-gauge (G) micropuncture needle. In some instances, the lateral dimension of the inner needle shaft 632 can be smaller than that of a standard gauge needle, including between that of a standard gauge needle and that of a micropuncutre needle, such as that of an 18-gauge (G) needle and the 22-gauge (G) micropuncture needle. In some instances, the lateral dimension of the inner needle shaft 632 can be between about 0.6 millimeters (mm) to about 1.2 millimeters (mm), including about 0.7 millimeters (mm) to about 1.1 millimeters (mm), and about 0.7 millimeters (mm) to about 1.0 millimeters (mm).

In some instances, the outer needle shaft 610 can have a uniform or substantially uniform lateral dimension, such as a diameter, along an entire or substantially entire longitudinal dimension, such as length, of the outer needle shaft 610. The lateral dimension can be perpendicular or substantially perpendicular to a longitudinal axis of the outer needle shaft 610. The longitudinal dimension can be a dimension parallel or substantially parallel to the longitudinal axis of the outer needle shaft 610. For example, the outer needle shaft 610 can have the same or similar outer diameter along an entire length extending distally of a distal end 608 of the outer needle hub 604, such as from a proximal end 612 to a distal end 614 of the outer needle shaft 610. In some instances, the distal end 614 can comprise at least a portion that is sharp and configured to pierce tissue to allow advancement of the outer needle shaft 610 through tissue, such as to allow navigation of the outer needle shaft 610 via a percutaneous access route. As described in further detail herein, the distal end 614 of the outer needle shaft 610 can be advanced into the pericardial space. The distal end 614 can be configured to pierce the pericardium to allow insertion of a portion of the outer needle shaft 610 into the pericardial space. In some instances, a sidewall 622 of the outer needle shaft 610 can define a shaft having the same or similar outer diameter along an entire or substantially entire length of the outer needle shaft 610 distal of the distal end 608 of the outer needle hub 604. In some instances, the outer needle shaft 610 can comprise a needle. In some instances, the lateral dimension, such as the outer diameter, of the outer needle shaft 610 can be the same as or similar to that of a standard gauge needle. In some instances, the outer needle shaft 610 can comprise a standard gauge needle. In some instances, the outer needle shaft 610 can comprise a lateral dimension that is the same as or similar to that of an 18-gauge (G) needle. The outer needle shaft 610 can comprise an 18-gauge (G) needle. In some instances, the lateral dimension of the outer needle shaft 610 can be larger than that of the standard gauge needle, such as the 18-gauge (G) needle. In some instances, the outer needle shaft 610 can comprise a lateral dimension, such as an outer diameter that is about 1.0 millimeters (mm) to about 1.6 millimeters (mm), including about 1.0 millimeters (mm) to about 1.5 millimeters (mm), and about 1.2 millimeters (mm) to about 1.4 millimeters (mm).

In some instances, the inner needle assembly 630 can be configured to be advanced relative to the outer needle assembly 602 to deploy the inner needle assembly 630. A portion of the inner needle shaft 632 can be configured to be disposed distally of a distal end 614 of the outer needle shaft 610 and positioned into a pericardial space. For example, the inner needle shaft 632 can be advanced relative to the outer needle shaft 610 to position a distal end opening 638 at a distal end 636 of the inner needle shaft 632 into the pericardial space. In some instances, the distal end 636 can comprise at least a portion that is sharp and configured to pierce tissue to allow advancement of the inner needle shaft 632 through tissue, such as to pierce the pericardium to allow advancement of the inner needle shaft 632 into the pericardial space. In some instances, the inner needle shaft 632 can be advanced until the distal end 654 of the inner needle hub 650 contacts the proximal end 606 of the outer needle hub 604. The distal end opening 638 can be in fluid communication with a delivery lumen 640 extending through the inner needle assembly 630. As described herein, positioning the distal end opening 638 of the inner needle shaft 632 within the pericardial space can be challenging. In the case that a portion of the inner needle shaft 632 is unintentionally advanced beyond the pericardial space, such as being deployed through the myocardium and into a heart chamber, such as a heart ventricle or atrium, the outer needle assembly 602 can be advanced relative to the inner needle assembly 630 to position a portion of the outer needle shaft 610 into the pericardial space. As described in further detail herein, if the inner needle shaft 632 overshoots the pericardial space, the position of the inner needle shaft 632 can be maintained while the outer needle shaft 610 is advanced relative to the inner needle shaft 632 to position a distal end opening 616 at the distal end 614 of the outer needle shaft 610 into the pericardial space. In some instances, a portion of a guidewire can be advanced through the second delivery lumen 620 and the distal end opening 616 into the pericardial space. After secure access to the pericardial space is achieved, medical therapy can be delivered through the inner needle shaft 632 or the outer needle shaft 610, including injection into and/or withdrawal from the pericardial space of any number of different fluids. In some instances, imaging contrast dye agents can be delivered.

In some instances, the outer needle hub 604 can comprise a first port 626 configured to mate with a first syringe (not shown). In some instances, the first port 626 can be in fluid communication with a delivery lumen of the outer needle assembly 602, such as the first and/or second delivery lumen 618, 620. For example, the first syringe can draw pericardial fluid from a pericardial space through the first and/or second delivery lumen 618, 620 and an access opening 628 of the first port 626 while the distal end opening 616 at the distal end 614 of the outer needle shaft 610 is positioned in the pericardial space. In some instances, a second syringe (not shown) can be mated with a second port 658, such as on a proximal end 652 of the inner needle hub 650, of the inner needle assembly 630 and configured to draw fluid through the delivery lumen 640 of the inner needle assembly 630. For example, the second syringe can draw blood through the delivery lumen 640 and second port 658 while the distal end opening 638 at the distal end 636 of the inner needle shaft 632 is positioned into the heart ventricle or atrium.

Alternatively, the second syringe can draw pericardial fluid through the delivery lumen 640 and second port 658 while the distal end opening 638 at the distal end 636 of the inner needle shaft 632 is positioned into the pericardial space. In some instances, one or more delivery lumens of the outer needle assembly 602 and/or inner needle assembly 630 can be used to deliver a solution to a target location, including a target location in the pericardial space. For example, contrast solution can be delivered to the pericardial space, such as for facilitating imaging procedures, through one or more of the delivery lumens. In some instances, one or more therapeutic agents can be delivered through the one or more delivery lumens.

In some instances, the inner needle shaft 632 can comprise an electrode 644 and the inner needle hub 650 can comprise an output contact 656 having one or more characteristics of the electrode 264 and the output contact 276 described with reference to FIG. 2, respectively. In some instances, the outer needle assembly 602, such as the outer needle hub 604, can comprise an advancement and retraction control 624 having one or more characteristics of the advancement and retraction control 222 described with reference to FIG. 2. In some instances, at least a portion of the outer needle shaft 610 and/or inner needle shaft 632 can be radiopaque. For example, the outer needle shaft 610 and the inner needle shaft 632 can be radiopaque to allow visualization of their respective positions, facilitating placement of the respective shafts. In some instances, radiopaque needle shafts having smaller diameters can be more difficult to visualize. The advancement and retraction control 624 can facilitate controlled advancement and/or retraction of the inner needle shaft 632 comprising a micropuncture needle.

In some instances, the outer needle assembly 602 can comprise an electrode (not shown) configured to provide an electrical signal indication when contact is made with a heart myocardium. For example, the outer needle shaft 610, including the distal end 614 of the outer needle shaft 610, can comprise an electrode having one or more characteristics of the electrode 264 described with reference to FIG. 2. The outer needle hub 604 can comprise an output contact (not shown) having one or more characteristics of the output contact 276 describe with reference to FIG. 2. In some instances, the inner needle assembly 630 can comprise one or more pressure sensors (not shown). For example, one or more pressure sensors can be associated with respective portions of the proximal portion, including the proximal end 634, and/or distal end 636 of the inner needle shaft 632. The pressure sensors can be coupled to, including mechanically and/or electrically, to a pressure sensor output on the inner needle hub 650. For example, the one or more pressure sensors and/or pressure sensor output can have one or more features as those described with reference to FIG. 2.

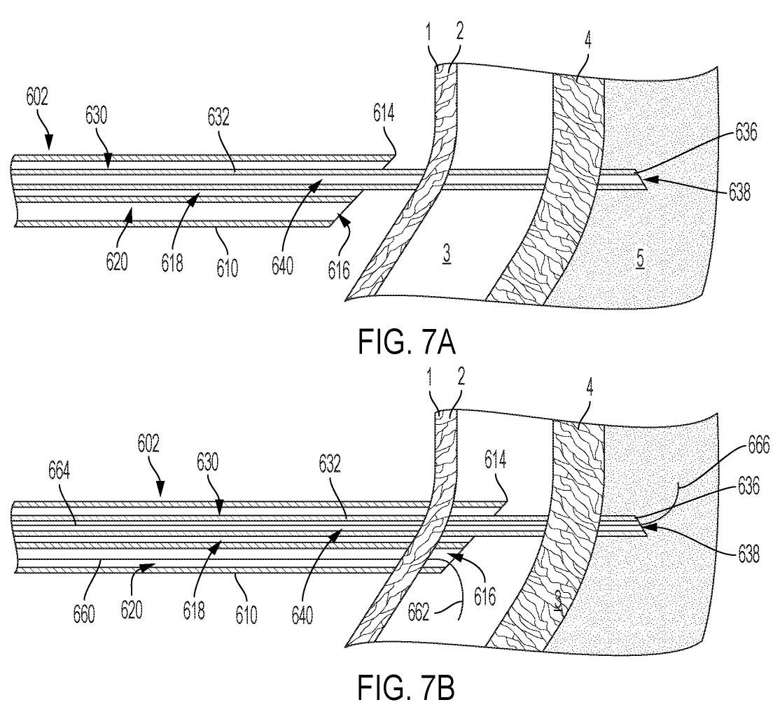

FIGS. 7A and 7B are side cross-sectional views of an example of accessing a heart pericardial space using the medical access system 600 described with reference to FIG. 6. FIG. 7A is a side cross-sectional view of the medical access system 600 having the inner needle shaft 632 positioned into a heart chamber 5. The heart chamber 5 can be a heart ventricle or a heart atrium. At least a portion of the inner needle shaft 632 can be preloaded within the outer needle assembly 602. For example, while the outer needle shaft 610 is advanced along an anatomical pathway, a portion of the inner needle shaft 632 can be within the second delivery lumen 620 and distal end 636 of the inner needle shaft 632 can be proximal of the distal end 614 of the outer needle shaft 610. The outer needle shaft 610 carrying the inner needle shaft 632 can be advanced to a target position, including for example positioning the distal end 614 of the outer needle shaft 610 proximate to or on an externally oriented surface of a pericardium 2 of a heart 1. The inner needle assembly 630 can be advanced to target positioning the distal end 636 of the inner needle shaft 632 through the pericardium 2 and into a pericardial space 3. As described herein, positioning the distal end 636 of the inner needle shaft 632 into the pericardial space 3 may be difficult. The inner needle shaft 632 can be advanced further than desired, overshooting the pericardial space 3. Referring to FIG. 7A, a portion of the inner needle shaft 632 can be positioned unintentionally through a myocardium 4 and into a heart chamber 5 of the heart 1.

Referring to FIG. 7B, in the case where the inner needle shaft 632 overshoots the intended target position in the pericardial space 3, the inner needle shaft 632 does not need to be retracted. In some instances, the inner needle shaft 632 can remain in place. The outer needle assembly 602 can be advanced to position the outer needle shaft 610 into the pericardial space 3. For example, the outer needle shaft 610 can be advanced relative to the inner needle shaft 632 such that the distal end opening 616 on the distal end 614 of the outer needle shaft 610 can be positioned into the pericardial space 3. Bleeding due to puncturing of the myocardium 5 by the inner needle shaft 632 can be reduced compared to that caused by larger bore needles. Advancement of the outer needle shaft 610 can be guided at least in part by the inner needle shaft 632.

In some instances, one or more guidewires can be deployed through a lumen of the outer needle assembly and/or the inner needle assembly, such as to facilitate secure positioning of the medical access system 600. For example, a first guidewire 660 can be deployed into the heart pericardial space 3 through the second delivery lumen 620 of the outer needle assembly 602. A portion of the first guidewire 660 can be advanced through the second delivery lumen 620 and the distal end opening 616 of the outer needle shaft 610 to be disposed in the pericardial space 3. For example, a distal portion 662 of the first guidewire 660 can be positioned into the pericardial space 3. In some instances, a second guidewire 664 can be deployed into the heart chamber 5 through the delivery lumen 640 of the inner needle assembly 630. In some instances, a portion of the second guidewire 664 can be advanced through the delivery lumen 640 of the inner needle assembly 630 and the distal end opening 638 at the distal end 636 of the inner needle shaft 632 to be positioned into the heart chamber 5. For example, a distal portion 666 of the second guidewire 664 can be positioned into the heart chamber 5. In some instances, at least a portion of the first guidewire 660 and/or second guidewire 664 can have a lateral dimension, such as a diameter, of about 0.25 millimeters (mm) to about 0.50 millimeters (mm), including about 0.35 millimeters (mm) to about 0.45 millimeters (mm). In some instances, at least a portion of the first guidewire 660 and/or second guidewire 664 can have a diameter of about 0.010 inches (in) to about 0.020 inches (in), including about 0.014 inches (in) to about 0.018 inches (in).

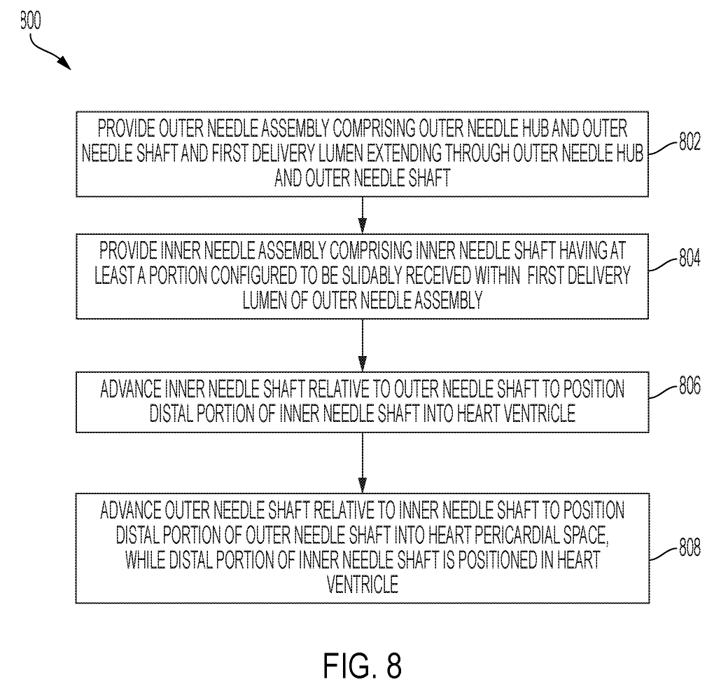

FIG. 8 is a process flow diagram illustrating an example of a procedure 800 for accessing a heart pericardial space. In block 802, the process can involve providing an outer needle assembly comprising an outer needle hub and an outer needle shaft extending distally from the outer needle hub. A first delivery lumen can extend through the outer needle hub and the outer needle shaft. In block 804, the process can involve providing an inner needle assembly comprising an inner needle shaft having at least a portion configured to be slidably received within the first delivery lumen of the outer needle assembly. In block 806, the process can involve advancing the inner needle shaft relative to the outer needle shaft to position a distal portion of the inner needle shaft into a heart ventricle. In alternative instances, the distal portion of the inner needle shaft can be positioned into a heart atrium. In block 808, the process can involve advancing the outer needle shaft relative to the inner needle shaft to position a distal portion of the outer needle shaft into the heart pericardial space, while the distal portion of the inner needle shaft is positioned in the heart ventricle. In some instances, the process can involve advancing the outer needle shaft relative to the inner needle shaft to position a distal portion of the outer needle shaft into the heart pericardial space, while the distal portion of the inner needle shaft is positioned in the heart atrium.

In some instances, the process can involve advancing the outer needle assembly to a target position. For example, advancing the outer needle assembly to a target position can comprise advancing the outer needle shaft along an anatomical pathway to position the distal end of the outer needle shaft at a desired position. In some instances, advancing the outer needle assembly to the target position can comprise positioning the distal end of the outer needle shaft proximate and/or adjacent to and/or adjacent to an outer surface of a pericardium. Advancing the outer needle assembly to the target position can comprise positioning the distal end of the outer needle shaft at a position spaced from and not in contact with the outer surface of the pericardium. Alternatively, advancing the outer needle assembly to the target position can comprise positioning the distal end of the outer needle shaft over and in contact with the outer surface of the pericardium.

In some instances, at least a portion of the inner needle shaft can be slidably disposed within the first delivery lumen of the outer needle assembly, for example preloaded within the first delivery lumen, while the outer needle assembly is advanced along the anatomical pathway. For example, the outer needle shaft can be advanced along an anatomical pathway to position the distal end of the outer needle shaft at the target position, while at least a portion of the inner needle shaft is preloaded through the first delivery lumen of the outer needle assembly. A distal end of the inner needle shaft can be within the first delivery lumen, such as proximal of the distal end of the outer needle shaft, while the inner needle shaft is preloaded within the outer needle assembly. Alternatively, the inner needle shaft can be advanced through the first delivery lumen of the outer needle assembly after the outer needle assembly is positioned at the target position.

In some instances, advancing the inner needle shaft relative to the outer needle shaft can comprise maintaining a position of the outer needle shaft while the inner needle shaft is deployed through a distal end opening at the distal end of the outer needle shaft. For example, the distal end of the outer needle shaft can be maintained at its position spaced from, or over and in contact with, the outer surface of the pericardium, while the inner needle shaft is deployed through the distal end opening of the outer needle shaft.

In some instances, the process can involve deploying a first guidewire through a second delivery lumen of the outer needle shaft into the heart pericardial space. For example, deploying the first guidewire can comprise advancing a portion of the first guidewire through the second delivery lumen and a distal end opening of the outer needle shaft into the heart pericardial space. In some instances, the process can involve deploying a second guidewire through a delivery lumen extending through the inner needle assembly into the heart ventricle or atrium. For example, deploying the second guidewire can comprise advancing a portion of the second guidewire through the delivery lumen and a distal end opening of the inner needle shaft into the heart ventricle or atrium. Positioning the first guidewire into the heart pericardial space and/or the second guidewire into the heart ventricle or atrium can facilitate secure positioning of the outer needle shaft and/or inner needle shaft.

In some instances, the process can involve mating a first syringe to a first port on the outer needle hub, the first port being in fluid communication with the first and/or second delivery lumen of the outer needle assembly to allow the first syringe to draw pericardial fluid from the heart pericardial space while the distal portion of the outer needle shaft is positioned in the heart pericardial space. In some instances, the process can involve mating a second syringe to a second port on the inner needle hub, the second port being in fluid communication with a delivery lumen extending through the inner needle assembly to allow the second syringe to draw blood from the heart ventricle or atrium while the distal portion of the inner needle shaft is positioned in the heart ventricle or atrium. In some instances, one or more delivery lumens of the inner needle assembly and/or outer needle assembly can be used to deliver one or more agents to a target location, such as a target location in the pericardial space. For example, contrast solution can be delivered to the pericardial space, such as for facilitating imaging procedures. In some instances, one or more therapeutic agents can be delivered.

### Description of Additional Examples

Provided below is a list of examples, each of which may include aspects of any of the other examples disclosed herein. Furthermore, aspects of any example described above may be implemented in any of the numbered examples provided below.

Example 1: A medical access system comprising an inner needle assembly including an inner needle shaft configured to be slidably disposed at least partially within a delivery lumen of an outer needle assembly. The inner needle shaft can include a narrower distal portion and a wider portion proximal of the narrower distal portion, and a transition segment between the narrower distal portion and the wider portion. The inner needle shaft can include first delivery lumen within the inner needle shaft extending from a proximal end of the inner needle shaft to a distal end opening at a distal end of the narrower distal portion, and a second delivery lumen within the inner needle shaft extending from the proximal end to a distal opening on the transition segment.

Example 2: The system of any example herein, in particular example 1, wherein the transition segment extends circumferentially around the inner needle shaft.

Example 3: The system of any example herein, in particular example 1 or 2, wherein the transition segment extends around a first circumferential portion of the inner needle shaft and a second circumferential portion of the inner needle shaft comprises a linear profile.

Example 4: The system of any example herein, in particular examples 1 to 3, wherein the transition segment comprises a tapered transition segment comprising a profile that tapers from the wider portion to the narrower distal portion.

Example 5: The system of any example herein, in particular example 4, wherein the distal opening on the transition segment is on the tapered transition segment.

Example 6: The system of any example herein, in particular examples 1 to 5, wherein the inner needle assembly is configured to be advanced relative to the outer needle assembly to, one of: position at least a portion of the narrower distal portion distally of a distal end of the outer needle assembly and into a heart pericardial space; or position the distal opening on the transition segment distally of the outer needle assembly and into a heart pericardial space.

Example 7: The system of any example herein, in particular examples 1 to 6, wherein the distal end of the inner needle shaft comprises an electrode configured to provide an electrical signal indication when the electrode contacts a heart myocardium.

Example 8: The system of any example herein, in particular examples 1 to 7, wherein the inner needle assembly comprises an inner needle hub and the inner needle shaft is configured to extend distally from the inner needle hub, and the inner needle hub comprises a first port configured to mate with a first syringe, the first port being in fluid communication with the first delivery lumen to allow the first syringe to draw pericardial fluid from a pericardial space while at least a portion of the narrower distal portion is positioned in the pericardial space or blood from a heart ventricle while at least a portion of the narrower distal portion is positioned in the heart ventricle.

Example 9: The system of any example herein, in particular examples 1 to 8, wherein the inner needle assembly comprises an inner needle hub and the inner needle shaft is configured to extend distally from the inner needle hub, and the inner needle hub comprises a second port configured to mate with a second syringe, the second port being in fluid communication with the second delivery lumen to allow the second syringe to draw pericardial fluid from a pericardial space while the distal opening on the transition segment is in the pericardial space.

Example 10: The system of any example herein, in particular examples 1 to 9, wherein the narrower distal portion of the inner needle shaft comprises a micropuncture needle.

Example 11: The system of any example herein, in particular examples 1 to 10, wherein the first needle shaft comprises an 18-gauge (G) needle.

Example 12: The system of any example herein, in particular examples 1 to 11, wherein the first delivery lumen and the second delivery lumen are each configured to slidably receive a guidewire having a diameter of about 0.014 inches (in) to about 0.018 inches (in).

Example 13: A method of accessing a heart pericardial space, the method comprising providing an outer needle assembly comprising an outer needle hub and an outer needle shaft extending distally from the outer needle hub and a delivery lumen extending through the outer needle hub and the outer needle shaft, and providing an inner needle assembly comprising an inner needle shaft having at least a portion configured to be slidably disposed within the delivery lumen of the outer needle assembly. The inner needle shaft can comprise a narrower distal portion, and a wider portion proximal of the narrower distal portion, and a transition segment between the narrower distal portion and the wider portion. The method can include advancing the inner needle shaft relative to the outer needle shaft to position at least a portion of the narrower distal portion distally of a distal end of the outer needle shaft.

Example 14: The method of any example herein, in particular example 13, wherein advancing the inner needle shaft relative to the outer needle shaft comprises positioning at least a portion of the narrower distal portion into the heart pericardial space.

Example 15: The method of any example herein, in particular example 13, wherein advancing the inner needle shaft relative to the outer needle shaft comprises positioning at least a portion of the narrower distal portion into a heart ventricle, and positioning a distal opening on the transition segment in the heart pericardial space.

Example 16: The method of any example herein, in particular examples 13 to 15, wherein providing the inner needle assembly comprises providing an inner needle assembly comprising a first delivery lumen within the inner needle shaft extending from a proximal end of the inner needle shaft to a distal end opening on a distal end of the narrower distal portion, and a second delivery lumen within the inner needle shaft extending from the proximal end to a distal opening on the transition segment.

Example 17: The method of any example herein, in particular example 16 further comprising advancing a portion of a first guidewire through the first delivery lumen and the distal end opening on the narrower distal portion into the heart pericardial space, while at least a portion of the narrower distal portion is positioned in the heart pericardial space.

Example 18: The method of any example herein, in particular example 16 further comprising advancing a portion of a second guidewire through the second delivery lumen and the distal opening on the transition segment into the pericardial space, while the distal opening on the transition segment is positioned in the heart pericardial space.

Example 19: The method of any example herein, in particular example 18, further comprising advancing a third guidewire through the first delivery lumen into the heart ventricle, while the distal opening on the transition segment is positioned in the heart pericardial space and the distal end opening on the distal end of the narrower distal portion is positioned in the heart ventricle.

Example 20: The method of any example herein, in particular example 16, wherein providing the inner needle assembly comprises providing an inner needle assembly comprising an inner needle hub and the inner needle shaft extending distally from the inner needle hub, and wherein the method further comprises mating a first syringe to a first port portion on the inner needle hub, the first port being in fluid communication with the first delivery lumen to allow the first syringe to draw pericardial fluid from the heart pericardial space while at least a portion of the narrower distal portion is positioned in the pericardial space or blood from a heart ventricle while at least a portion of the narrower distal portion is positioned in the heart ventricle.

Example 21: The method of any example herein, in particular example 16, wherein providing the inner needle assembly comprises providing an inner needle assembly comprising an inner needle hub and the inner needle shaft extending distally from the inner needle hub, and wherein the method further comprises mating a second syringe to a second port on the inner needle hub, the second port being in fluid communication with the second delivery lumen to allow the second syringe to draw pericardial fluid from a pericardial space while the distal opening on the transition segment is positioned in the pericardial space.

Example 22: The method of any example herein, in particular examples 13 to 21, further comprising contacting an electrode at the distal end of the narrower distal portion to a heart myocardium.

The above method(s) can be performed on a living animal or on a simulation, such as on a cadaver, cadaver heart, anthropomorphic ghost, simulator (e.g., with body parts, heart, tissue, etc. being simulated).

Example 23: A method of accessing a heart pericardial space, the method comprising providing an outer needle assembly comprising an outer needle hub and an outer needle shaft extending distally from the outer needle hub and a first delivery lumen extending through the outer needle hub and the outer needle shaft, providing an inner needle assembly comprising an inner needle shaft having at least a portion configured to be slidably received within the first delivery lumen of the outer needle assembly, advancing the inner needle shaft relative to the outer needle shaft to position a distal portion of the inner needle shaft into a heart ventricle, and advancing the outer needle shaft relative to the inner needle shaft to position a distal portion of the outer needle shaft into the heart pericardial space, while the distal portion of the inner needle shaft is positioned in the heart ventricle.

Example 24: The method of any example herein, in particular example 23 further comprising advancing a portion of a first guidewire through a second delivery lumen of the outer needle shaft into the heart pericardial space.

Example 25: The method of any example herein, in particular example 23 or 24 further comprising advancing a portion of a second guidewire through a delivery lumen extending through the inner needle assembly to position the second guidewire into the heart ventricle.

Example 26: The method of any example herein, in particular examples 23 to 25 further comprising mating a first syringe to a first port on the outer needle hub, the first port being in fluid communication with at least one of the first delivery lumen and a second delivery lumen of the outer needle assembly to allow the first syringe to draw pericardial fluid from the heart pericardial space while the distal portion of the outer needle shaft is positioned in the heart pericardial space.

Example 27: The method of any example herein, in particular examples 23 to 26, wherein providing the inner needle assembly comprises providing an inner needle assembly comprising an inner needle hub and the inner needle shaft extending distally from the inner needle hub, and wherein the method further comprises mating a second syringe to a second port on the inner needle hub, the second port being in fluid communication with a delivery lumen extending through the inner needle assembly to allow the second syringe to draw blood from the heart ventricle while the distal portion of the inner needle shaft is positioned in the heart ventricle.

The above method(s) can be performed on a living animal or on a simulation, such as on a cadaver, cadaver heart, anthropomorphic ghost, simulator (e.g., with body parts, heart, tissue, etc. being simulated).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other instance(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of accessing a pericardial space of a heart, the method comprising:
advancing at least a portion of an outer needle assembly to an outer surface of a pericardium of a heart, the outer needle assembly comprising an outer needle shaft and a delivery lumen extending through the outer needle shaft;
providing an inner needle assembly comprising an inner needle shaft having at least a portion slidably disposed within the delivery lumen of the outer needle assembly, the inner needle shaft comprising a narrower distal portion having a distal opening at a distal end of the narrower distal portion, a wider portion proximal of the narrower distal portion, and a transition segment between the narrower distal portion and the wider portion, the transition segment comprising a proximal opening;
advancing the inner needle shaft relative to the outer needle shaft and through the outer surface of the pericardium to position at least a portion of the transition segment including the proximal opening within the pericardial space; and
advancing a first guidewire through the inner needle shaft and out of the proximal opening into the pericardial space.

2. The method of claim 1, further comprising:
advancing the narrower distal portion through the outer surface of the pericardium to position the distal opening within the pericardial space; and
advancing a second guidewire through the inner needle shaft and out of the distal opening into the pericardial space.

3. The method of claim 1, further comprising advancing at least a portion of the inner needle shaft through the outer surface of the pericardium to position at least the distal end of the narrower distal portion within a heart ventricle.

4. The method of claim 1, wherein the outer needle assembly further comprises an outer needle hub, wherein the outer needle shaft extends distally from the outer needle hub, and wherein the delivery lumen extends through the outer needle hub and the outer needle shaft.

5. The method of claim 1, wherein the inner needle assembly comprises a first delivery lumen extending from a proximal end of the inner needle shaft to a distal end opening on a distal end of the narrower distal portion and a second delivery lumen extending from the proximal end to the proximal opening.

6. The method of claim 1, wherein the inner needle assembly comprises an inner needle hub, and wherein the inner needle shaft extends distally from the inner needle hub, the method further comprising:
mating a first syringe to a first port portion on the inner needle hub, the first port portion being in fluid communication with the inner needle shaft to allow the first syringe to draw pericardial fluid from the pericardial space while at least a portion of the narrower distal portion is positioned in the pericardial space or blood from a heart ventricle while at least a portion of the narrower distal portion is positioned in the heart ventricle; or
mating a second syringe to a second port on the inner needle hub, the second port being in fluid communication with the inner needle shaft to allow the second syringe to draw pericardial fluid from the pericardial space while the proximal opening is positioned in the pericardial space.

7. The method of claim 1, further comprising contacting an electrode at a distal end of the narrower distal portion to a heart myocardium.

8. A method of accessing a pericardial space of a heart, the method comprising:
advancing at least a portion of an outer needle assembly to an outer surface of a pericardium of a heart, the outer needle assembly comprising an outer needle shaft and a delivery lumen extending through the outer needle shaft;
providing an inner needle assembly comprising an inner needle shaft having at least a portion configured to be slidably disposed within the delivery lumen of the outer needle assembly, and the inner needle shaft comprising a narrower distal portion, a wider portion proximal of the narrower distal portion, and a transition segment between the narrower distal portion and the wider portion, the transition segment comprising a proximal opening; and
advancing the inner needle shaft relative to the outer needle shaft and through the outer surface of the pericardium to position at least a portion of the transition segment including the proximal opening within the pericardial space.

9. The method of claim 8, wherein the inner needle assembly comprises a distal opening at a distal end of the narrower distal portion.

10. The method of claim 9, further comprising advancing the narrower distal portion through the outer surface of the pericardium to position the distal opening within the pericardial space.

11. The method of claim 10, further comprising advancing a first guidewire through the inner needle shaft and out of the distal opening into the pericardial space.

12. The method of claim 11, further comprising advancing a second guidewire through the inner needle shaft and out of the proximal opening into the pericardial space.

13. The method of claim 8, further comprising advancing a second guidewire through the inner needle shaft and out of the proximal opening into the pericardial space.

14. The method of claim 8, further comprising advancing at least a portion of the inner needle shaft through the outer surface of the pericardium to position a distal end of the narrower distal portion within a heart ventricle.

15. The method of claim 8, wherein the outer needle assembly further comprises an outer needle hub, wherein the outer needle shaft extends distally from the outer needle hub, and wherein the delivery lumen extends through the outer needle hub and the outer needle shaft.

16. The method of claim 8, wherein the inner needle assembly comprises a first delivery lumen extending from a proximal end of the inner needle shaft to a distal end opening on a distal end of the narrower distal portion and a second delivery lumen extending from the proximal end to the proximal opening.

17. The method of claim 8, wherein the inner needle assembly comprises an inner needle hub, and wherein the inner needle shaft extends distally from the inner needle hub, the method further comprising:
mating a first syringe to a first port portion on the inner needle hub, the first port portion being in fluid communication with the inner needle shaft to allow the first syringe to draw pericardial fluid from the pericardial space while at least a portion of the narrower distal portion is positioned in the pericardial space or blood from a heart ventricle while at least a portion of the narrower distal portion is positioned in the heart ventricle; or
mating a second syringe to a second port on the inner needle hub, the second port being in fluid communication with the inner needle shaft to allow the second syringe to draw pericardial fluid from the pericardial space while the proximal opening is positioned in the pericardial space.

18. The method of claim 8, further comprising contacting an electrode at a distal end of the narrower distal portion to a heart myocardium.

19. A method of accessing a pericardial space of a heart, the method comprising:
advancing at least a portion of an outer needle assembly to an outer surface of a pericardium of a heart, the outer needle assembly comprising an outer needle shaft and a delivery lumen extending through the outer needle shaft;
providing an inner needle assembly comprising an inner needle shaft having at least a portion configured to be slidably disposed within the delivery lumen of the outer needle assembly, and the inner needle shaft comprising a narrower distal portion, a wider portion proximal of the narrower distal portion, and a transition segment between the narrower distal portion and the wider portion, the transition segment comprising a proximal opening, the inner needle assembly comprising a first delivery lumen extending from a proximal end of the inner needle shaft to a distal end opening on a distal end of the narrower distal portion and a second delivery lumen extending from the proximal end to the proximal opening; and
advancing the inner needle shaft relative to the outer needle shaft and through the outer surface of the pericardium to position at least a portion of the transition segment including the proximal opening within the pericardial space.

20. The method of claim 19, further comprising advancing a first guidewire through the inner needle shaft and out of the proximal opening into the pericardial space.
